(19) United States
(12) Patent Application Publication
(10) Pub. No.: US 2013/0014764 A1
(43) Pub. Date: Jan. 17, 2013

(54) Foreskin Retaining Device

(71) Applicant: Aleq Rojas, Sahuarita, AZ (US)

(72) Inventor: Aleq Rojas, Sahuarita, AZ (US)

(21) Appl. No.: 13/539,085

(22) Filed: Jun. 29, 2012

Related U.S. Application Data
(60) Provisional application No. 61/508,515, filed on Jul. 15, 2011.

(51) Int. Cl. A61F 6/04

(52) U.S. Cl. 128/844

(57) ABSTRACT

A foreskin-retaining device for an intact penis improves upon known devices by including a retaining section on an inner surface of the device, the retaining section having protrusions for contacting and holding the retracted foreskin. The protrusions may be rings, nodes, dull barbs, or a combination thereof. An adhesive may also be applied in the retaining section. The retaining section may be slightly wider than the rest of the device to provide the space needed to hold the loose skin. The device may have the characteristics of a condom, such as a flared head and reservoir tip, or may be used in conjunction with a condom for contraceptive purposes.

### CROSS-REFERENCE TO RELATED APPLICATIONS

This application is a nonprovisional and claims the benefit of U.S. Pat. Ser. No. 61/508,515, filed Jul. 15, 2011.

FIELD OF INVENTION

This invention relates to condoms. This invention relates particularly to a device for retaining the foreskin of an uncircumcised penis in a retracted position.

### BACKGROUND

Male circumcision is the process of removing the foreskin of a male's penis. While male circumcision is a common practice, many men do not have the foreskin removed from the penis. An uncircumcised, or “intact,” penis has a significant amount of additional skin. The foreskin partially retracts as the penis becomes erect, but the foreskin does not become completely tight. Instead, the loose skin can slide along the shaft during intercourse. The sliding causes a significant problem for a man wishing to use a condom during intercourse. Known condoms are designed to fit tightly and remain in place during intercourse, but on an intact penis, the sliding action of the loose skin between the penis and the condom makes keeping the condom in place difficult or impossible. A condom misused in this manner may not protect against unwanted pregnancy or sexually-transmitted diseases, and further may cause injury to the man or his partner. A contraception device specially designed for an intact penis is needed.

One known contraception device for an intact penis uses two rings positioned near the base of a condom and spaced apart to retain the foreskin between them. This design lacks flexibility, in that variations among men related to size or amount of foreskin may impact or negate the effectiveness of the two rings. A contraception device that is effective across such varying dimensions is needed.

Therefore, it is an object of this invention to provide a device for intact penises that secures the foreskin to keep the device in place. It is a further object that the device be usable by men having various penis sizes and amounts of foreskin.

### SUMMARY OF THE INVENTION

The present invention is a device for retaining the foreskin of an intact penis in a retracted position. The device may have many of the features typical for known condoms, including a thin-walled body with a flared or bulb-shaped head and reservoir tip at the proximal end and a substantially elastic base ring at the distal end. The device further has a retaining section which secures the foreskin in a retracted position and keeps the device in place. The retaining section is a section of the device adjacent to the base ring that holds the loose skin in place inside the device. The retaining section secures the foreskin using a plurality of contoured protrusions on the inner surface. In one embodiment, the protrusions are annular rings. The annular rings may have a smooth surface, or may have jagged edges or secondary protrusions to increase friction between the protrusions and the skin. In another embodiment, the protrusions are a series of nodes spaced about the inner surface. In another embodiment, the device includes multiple different types of protrusions arranged beneficially. Additionally, the retaining section may be slightly wider than the rest of the device to provide the space needed to hold the loose skin.

### BRIEF DESCRIPTION OF THE DRAWINGS

FIG. 1 is a perspective view of a first embodiment of the invention illustrating ring protrusions.

FIG. 2 is a perspective view of a second embodiment of the invention illustrating jagged ring protrusions.

FIG. 3 is a perspective view of a third embodiment of the invention illustrating wavy ring protrusions.

FIG. 4 is a perspective view of a fourth embodiment of the invention illustrating radial node protrusions.

FIG. 5 is a rear perspective view of a fifth embodiment of the invention illustrating a combination of different types of protrusions.

FIG. 6 is a perspective view of a mold used to form one or more of the embodiments illustrated in FIGS. 1-3.

FIG. 7 is a perspective view of a sixth embodiment of the invention illustrating jagged ring protrusions and a proximal ring.

FIG. 8 is a perspective view of a seventh embodiment of the invention illustrating node protrusions and no base or proximal ring.

FIG. 9 is a perspective view of an eighth embodiment of the invention illustrating node protrusions and a buckle.

### DETAILED DESCRIPTION OF THE INVENTION

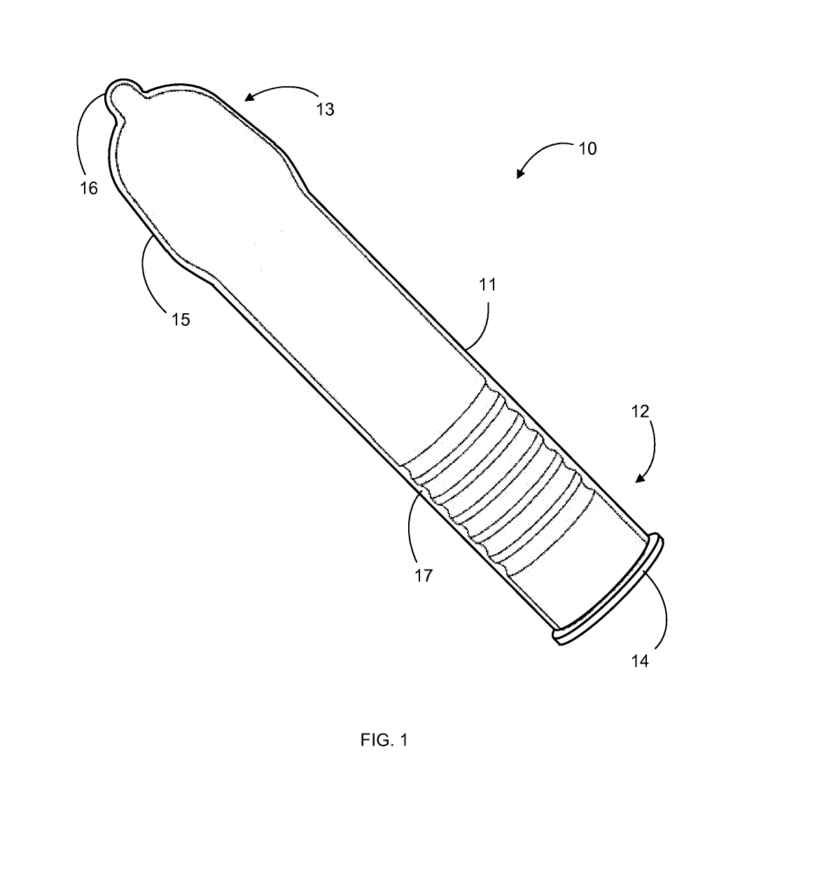

Referring to FIGS. 1-5, there are illustrated several embodiments of the present invention, designated generally as 10, which is a foreskin retaining device for use on an intact penis. The device 10 has a flexible body 11 made of any material known in the art to be suitable for condoms, such as latex, polyurethane, or lambskin. The body 11 has a distal end 12 and a proximal end 13. The body 11 further has an inner surface, which is the surface intended to contact the penis during use, and an outer surface, which contacts the environment. A base ring 14 may be disposed around the body 11 at the distal end 12. The base ring 14 is designed to fit tightly around the shaft of the wearer's penis to retain the device 10 on the shaft, as is known in the art. The base ring 14 may be adhesively or non-adhesively attached to the body 11, but is preferably integral with the body 11. The proximal end 13 may include a portion of the body 11 that has a larger diameter than the main part of the body 11, forming a flared head 15 designed to fit around the glans of the penis. The device 10 may further have a reservoir tip 16 for reducing fluid pressure of collected semen, as is known in the art. The device 10 may have other known features of condoms, such as lubricated (standard or spermicidal) surfaces and external texturing.

The device 10 is improved over existing condoms for use on an intact penis because it has a retaining section 20 incorporated into the inner surface of the body 11. The retaining section 20 secures the foreskin in a retracted position by friction, adhesive, or barrier, or a combination of those. The securing keeps the device 10 in place during intercourse or other use. The retaining section 20 may have a series of protrusions 17 disposed on the inner surface of the body 11 in a portion of the device 10 near or adjacent to the base ring 14. The section of the device 10 measured from the most distal protrusion 17 to the most proximal protrusion 17 is the retaining section 20. The protrusions 17 are preferably integral with, and most preferably molded into, the body 11 at the inner surface. The protrusions 17 are thicker and firmer than the main part of the body 11, fitting tighter in contact with the shaft, or with the foreskin when the foreskin is retracted. The protrusions thereby hold the foreskin in a retracted position using friction or by serving as a barrier to the foreskin's passage, and further keep the device 10 from sliding or rotating with respect to the shaft insofar as one or more protrusions 17 are in contact with the shaft. Specifically, of the plurality of protrusions 17, at least one of the protrusions 17 contacts the retracted foreskin, applying enough force so that the foreskin does not slide proximally or distally with respect to the protrusion 17 contacting it. All of the protrusions 17 may contact the foreskin, but preferably the protrusions 17 are spaced so that one or more of the protrusions 17 may contact the shaft proximally or distally of the retracted foreskin to further hold the device 10 in place. Alternatively, the part of the retaining section 20 that contacts the shaft may include an adhesive on the inner surface. Three example embodiments are illustrated in FIGS. 1-3 and described below.

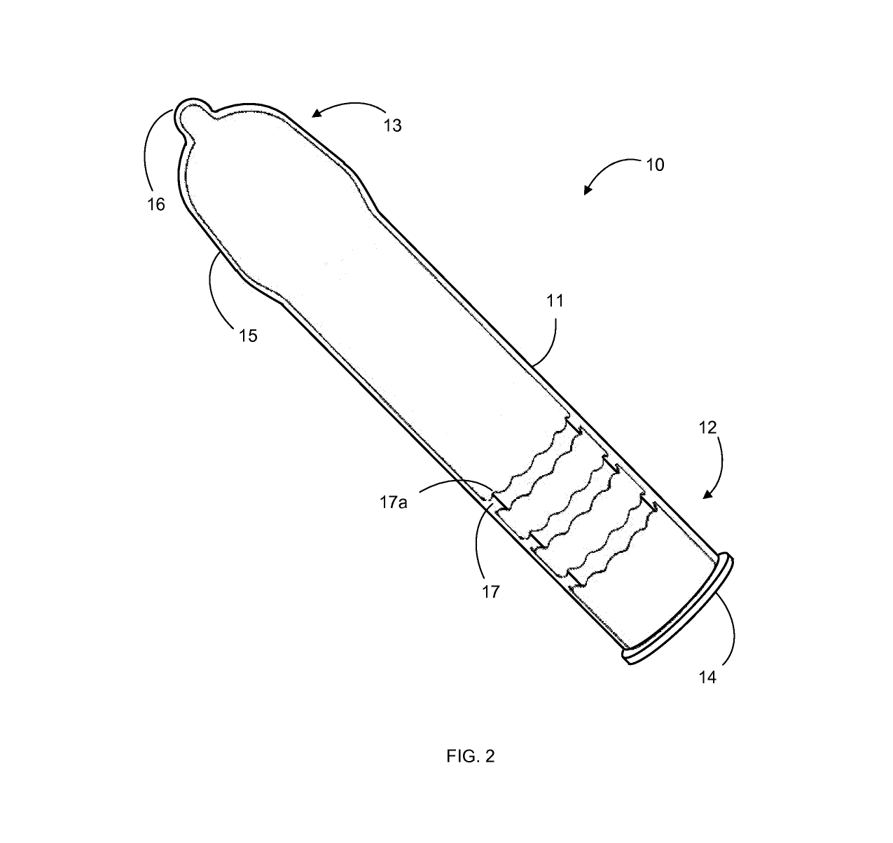

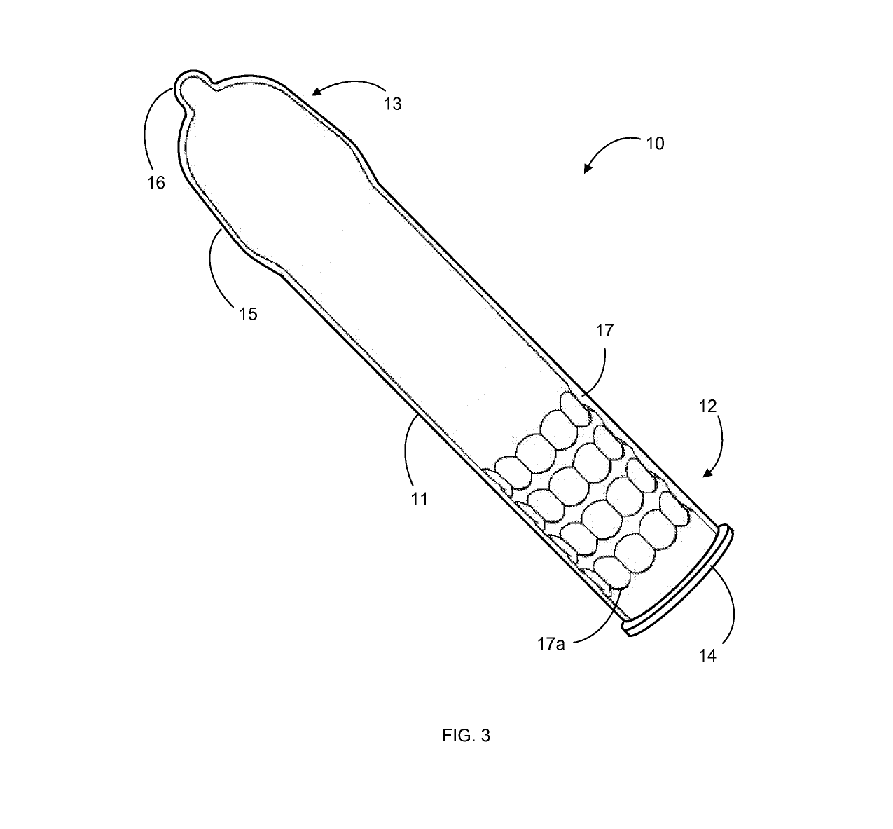

As shown in FIGS. 1-3, the protrusions 17 may be three or more annular rings that span the circumference of the body 11. The greater the number of rings, the better the device 10 will retain the foreskin in a retracted position while stabilizing the device 10 in position with respect to the shaft. The protrusions 17 may be uniformly or non-uniformly spaced along the inner surface of the body 11. The height of the protrusions 17, measured radially inward from the inner surface of the body 11, may be uniform or non-uniform between the protrusions 17, and further may be constant or varied on a single protrusion 17. The height variation on a protrusion 17 may be in any direction, such as proximal to distal, giving the protrusion 17 a slope, or circumferential, giving the protrusion 17 a wavy or jagged inner circumference. The width of a protrusion 17, measured proximally to distally, may be uniform or, as illustrated in FIGS. 2 and 3, may vary, giving the protrusion 17 jagged or wavy sides 17a. The cross-section of the protrusions 17, taken along a plane in which the axis of the device 10 lies, may be rounded, triangular, square, rectangular, or irregularly shaped. The cross-section may be uniform throughout, or may change in scale or shape.

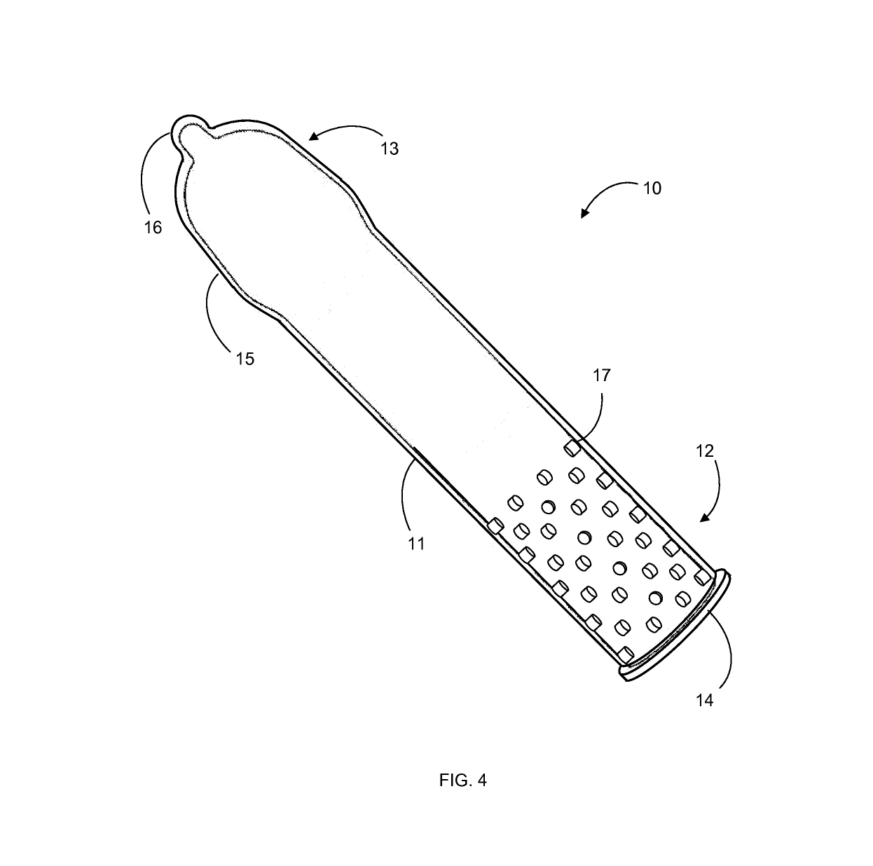

Referring to FIG. 4, other embodiments of the device 10 may include protrusions 17 comprising a series of nodes spread circumferentially and axially on the inside surface of the body 11. As with the rings of FIGS. 1-3, the height and width of each node may be uniform or variable with respect to other nodes as well as within the node itself. A node may have any shape and cross-section suitable for providing friction against the skin, including circular, oval, squared, rectangular, or polygonal shapes, X shapes, or cross shapes, with curved, squared, rectangular, or irregular cross-sections. The nodes may be arranged in any suitable pattern, with any suitable spacing between the nodes. The nodes may project substantially radially inward, or may project at an angle offset from radial, toward either the proximal or distal ends of the body 11, or further may present in a combination of radial and angled orientations. A combination of radially-projecting and angled nodes is preferred, with the angle of offset also varying, in order to produce a field of nodes that is not susceptible to shift in any particular direction. Alternatively, the nodes may all be angled toward the proximal end of the body 11 in order to provide little resistance to sliding in the distal direction but high resistance to sliding in the proximal direction.

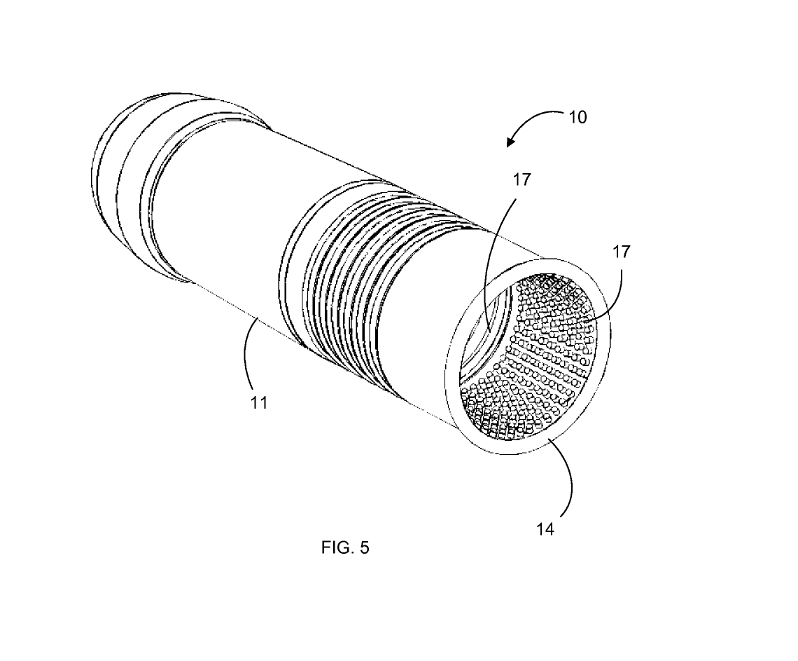

Referring to FIG. 5, the retaining section 20 may alternatively include a plurality of types of protrusions 17, such as a mixture of nodes and rings, in any advantageous combination. A protrusion 17 may further have one or more smaller secondary protrusions or a rough texture on the surface of the protrusion 17 that contacts the skin, in order to increase the friction between the skin and the device 10 to hold the device 10 in place. Additionally, the retaining section 20 may be slightly wider than the rest of the device 10 to provide space between the inner surface of the body 11 and the penis, which space may be used to hold the loose skin. It will be understood that in such an embodiment, the protrusions 17 may need to be taller to accommodate the widening of the body 11 at the retaining section 20.

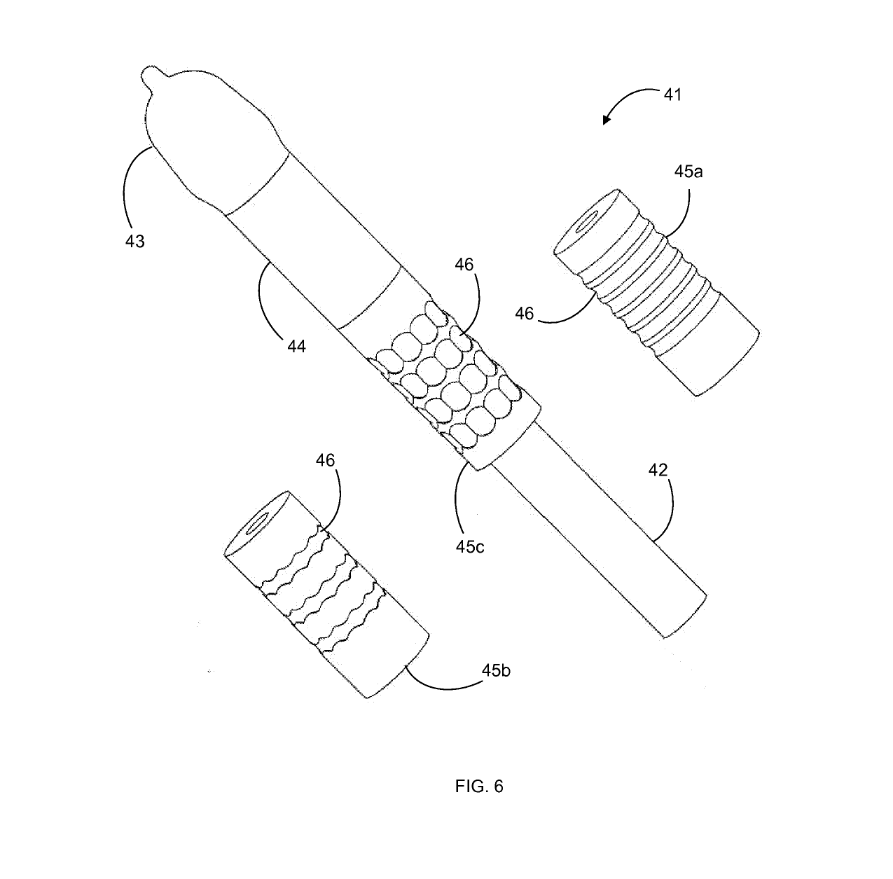

Referring to FIG. 6, the device 10 may be molded using a mold 41 having cavities 46 that define the protrusions 17 when the device 10 is molded. The mold 41 may be a single, integral unit, or may have one or more interchangeable sections that allow different configurations of the device 10 to be molded. For example, the mold 41 may comprise a proximal mold 43 defining features such as the head 15 and reservoir tip 16; a middle mold 44 defining the main part of the body 11; and a distal mold 45a, 45b, 45c having the cavities 46 to form the protrusions 17 of the retaining section 20, and further defining the base ring 14, if any. The interchangeable sections are mounted on a mounting rod 42. In the illustrated example, distal mold 45a fabricates the device 10 of FIG. 1, distal mold 45b fabricates the device 10 of FIG. 2, and distal mold 45c fabricates the device 10 of FIG. 3.

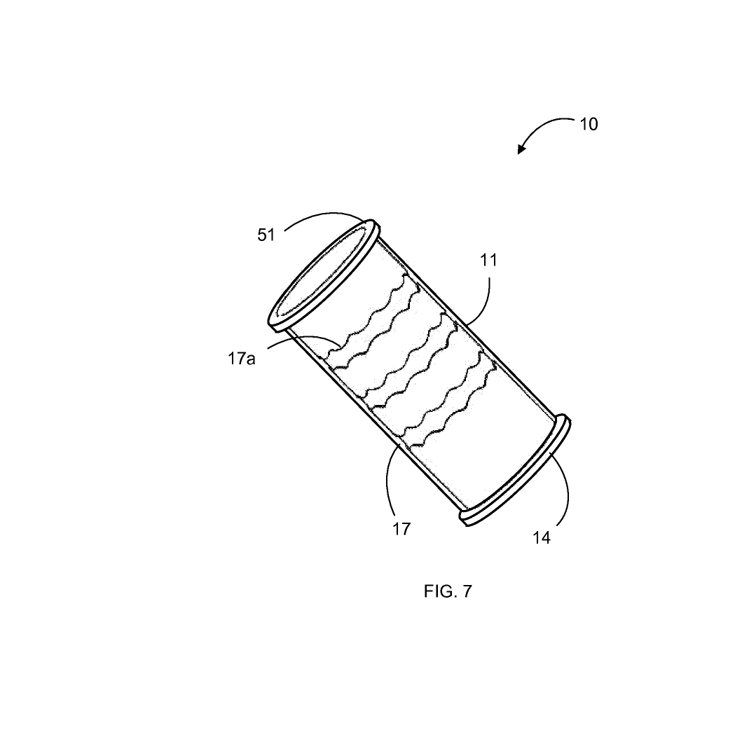

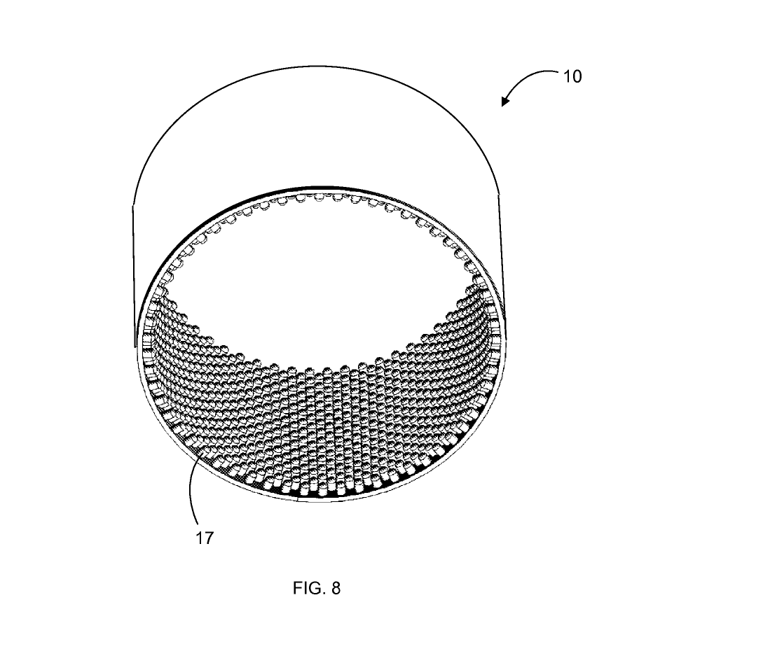

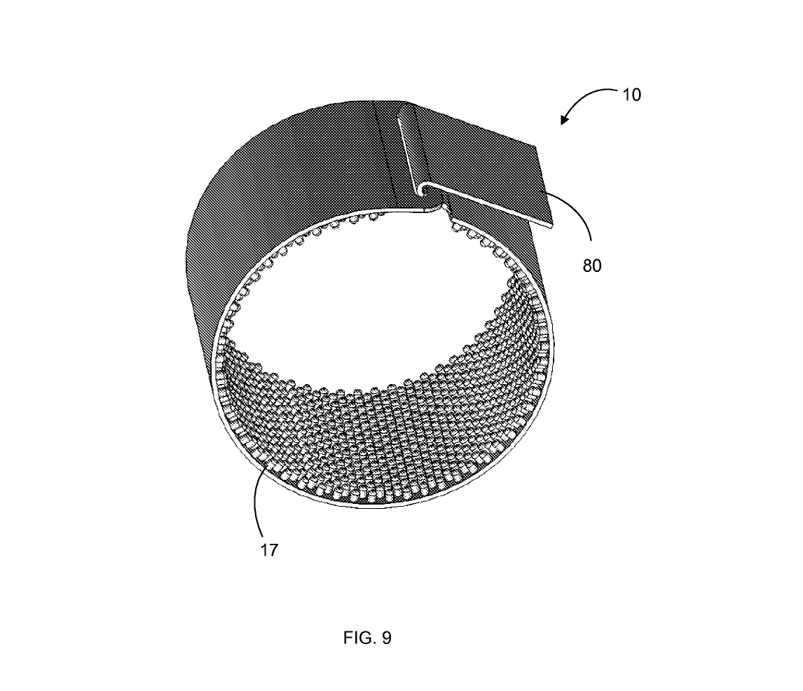

In other embodiments, illustrated in FIGS. 7-9, the device 10 may encircle only a portion of the shaft that includes the retracted foreskin, thus not spanning the shaft or encasing the glans. In this embodiment, the device 10 serves no contraceptive purpose. However, the device may be made thin enough that a condom may be placed over the device 10 if desired. In the embodiment shown in FIG. 6, the device 10 is held in place by the base ring 14, the protrusions 17, and a proximal ring 51 attached to the body 11 at the proximal end. The proximal ring 51 serves a similar purpose to a ring protrusion 17 described above, so that the retaining section 20 extends from the furthest distal protrusion 17 to the proximal ring 51. In other embodiments, one or both of the base ring 14 and proximal ring 51 may be absent, and the device 10 may be held in place by the protrusions 17, see FIG. 7, or by an adhesive applied to the protrusions 17 and the inner surface of the body 11. In still other embodiments, the device 10 may be a strip that is wrapped around the shaft and retracted foreskin and secured in place by an adhesive. In embodiments using an adhesive, the adhesive is preferably bio-compatible and may further be warming to enhance sensations. In still another embodiment, shown by example in FIG. 8, the device 10 may be a strip that is wrapped around the shaft and retracted foreskin and secured in place by an adjustable buckle 80. The buckle 80 may be held closed by an adhesive, or by VELCRO® or another hook-and-loop fabric.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A foreskin-retaining device for use on an intact penis, the device comprising:
a. a body having a proximal end, a distal end, an outer surface, and an inner surface, the body being configured to encircle at least a portion of the penis' foreskin so that the inner surface contacts the foreskin when the foreskin is retracted; and
b. a retaining section positioned on the inner surface of the body where the inner surface contacts the foreskin, the retaining section comprising one or more protrusions that contact the foreskin to retain the foreskin in its retracted position.

2. The device of claim 1 wherein the retaining section extends to a midpoint between the proximal and distal ends of the body.

3. The device of claim 2 comprising a plurality of protrusions each comprising a node.

4. The device of claim 3 wherein at least one of the nodes extends radially from the inner surface.

5. The device of claim 3 wherein at least one of the nodes extends from the inner surface at an angle offset from radial.

6. The device of claim 4 wherein a plurality of nodes extend from the inner surface at differing angles offset from radial.

7. The device of claim 6 wherein the nodes are arranged in adjacent circles around the circumference of the inner surface.

8. The device of claim 7 wherein the circles of nodes extend from the midpoint between the proximal and distal ends of the body to the distal end of the body.

9. The device of claim 1 wherein the retaining section further comprises adhesive.

10. The device of claim 9 wherein the adhesive is applied to the protrusions.

11. The device of claim 9 wherein the adhesive is applied to the inner surface between the protrusions.

12. The device of claim 1 wherein at least one of the protrusions contacts the shaft of the penis proximal to the foreskin.

13. The device of claim 12 wherein at least one of the protrusions that contacts the shaft also contacts the foreskin to act as a barrier against the foreskin's proximal-to-distal movement.

14. The device of claim 13 wherein the portion of the body having the retaining section on the inner surface has a larger circumference than the other portions of the body, the larger circumference aiding in retaining the foreskin within the retaining section.

15. The device of claim 1 wherein the protrusions are further configured to retain the device in a particular position during intercourse.

16. The device of claim 1 further comprising a base ring attached to the distal end of the body and configured to fit tightly around the base of the penis to aid in retaining the device in place during intercourse.

17. The device of claim 16 wherein the retaining section is adjacent to the base ring.

18. The device of claim 16 wherein the retaining section is spaced proximally away from the base ring.

19. The device of claim 16 further comprising a proximal ring attached to the proximal end of the body and configured to fit tightly around the shaft of the penis proximally from the retracted foreskin.

20. The device of claim 1 further comprising a buckle attached to the body, the body encircling the portion of the penis' foreskin when the buckle is closed.
